(19) United States
(12) Patent Application Publication
(10) Pub. No.: US 2024/0203583 A1
(43) Pub. Date: Jun. 20, 2024

(54) METHOD FOR FLAGGING SURGICAL INFORMATION WITH METADATA

(71) Applicant: Covidien LP, Boulder, CO (US)

(72) Inventor: Scott Frushour, Boulder, CO (US)

(73) Assignee: Covidien LP, Boulder, CO (US)

(21) Appl. No.: 18/535,054

(22) Filed: Dec. 11, 2023

Related U.S. Application Data
(60) Provisional application No. 63/476,183, filed on Dec. 20, 2022.

(51) Int. Cl. G16H 40/67; G16H 10/00

(52) U.S. Cl. CPC G16H 40/67; G16H 10/00

(57) ABSTRACT

This document discloses system and method embodiments for flagging surgical information with metadata. For example, the method includes, by a processor of an intelligent medical device, acquiring data from one or more sensors of an intelligent medical device. The method further includes receiving a signal from a user, the signal indicating a moment in time during a medical procedure. The method further includes flagging the acquired data with metadata based on the time of the received signal and storing the acquired data and the metadata on a server.

### CROSS REFERENCE TO RELATED APPLICATIONS

This application claims priority under 35 U.S.C. § 119(e) to U.S. Provisional Application No. 63/476,183, filed Dec. 20, 2022, the disclosure of which is incorporated herein by reference in its entirety.

### FIELD

The present technology is generally related to systems and methods for robot-assisted medical procedures.

### BACKGROUND

Surgical and interventional procedures are increasingly complex. In the case of robotically assisted minimally-invasive procedures, the surgeon may use telemanipulation, e.g., operating surgical tools or instruments through computer control. In the case of image-guided surgery (IGS) the surgeon may track the surgical instruments in conjunction with preoperative or intraoperative images, e.g., using cameras, ultrasonic, electromagnetic, or other imaging techniques. Measurement and recording techniques that are not primarily designed to produce images, such as electroencephalography (EEG), magnetoencephalography (MEG), electrocardiography (ECG), and others, represent other technologies that produce data susceptible to representation as a parameter graph versus time or maps that contain data about the measurement locations. Synchronizing these potentially independent data streams, in light of the high aggregate data volume of these streams, presents challenges in general. It can be particularly challenging to identify, after the fact, a point in time during a procedure that is of particular interest and to locate that point in time in each recorded data streams.

This document describes methods and systems that are directed to addressing the problems described above, and/or other issues.

### SUMMARY

The techniques of this disclosure generally relate to flagging surgical information with metadata. At least some of the problems associated with the existing solutions will be shown solved by the subject matter of the independent claims that are included in this document. Additional advantageous aspects are discussed in the dependent claims.

In one aspect, the present disclosure provides a method including, by a processor of an intelligent medical device, acquiring data from one or more sensors of an intelligent medical device. The method further includes receiving a signal from a user, the signal indicating a moment in time during a medical procedure. The method further includes flagging the acquired data with metadata based on the time of the received signal and storing the acquired data and the metadata on a server.

Implementations of the disclosure may include one or more of the following optional features. In some examples, acquiring the data from the one or more sensors of the intelligent medical device includes acquiring a stream of data from the one or more sensors. In some examples, acquiring the data from the one or more sensors includes acquiring the data from multiple sensors. Receiving the signal from the user may include receiving input from a floor pedal. Flagging the acquired data with metadata may include flagging the acquired data with metadata indicating a start time and a stop time based on the time of the received signal. In some examples, the method further includes retrieving the stored data and presenting the stored data for review based on the metadata.

In another aspect, the disclosure provides an intelligent medical device including one or more sensors configured to generate a stream of digital data during a medical procedure. The system further includes an input device and includes a processor and a computer-readable memory containing programming instructions. The programming instructions are configured to, when executed by the processor, cause the processor to receive a signal from the input device. The programming instructions are further configured to cause the processor to flag the stream of digital data with metadata indicating a time of the received signal and store the acquired data and the metadata on a server for subsequent review.

Implementations of the disclosure may include one or more of the following optional features. In some examples, the one or more sensors include a camera. The intelligent medical device may further include a surgical robot. In some examples, the input device includes an audio sensor and the programming instructions are configured to cause the processor to receive input from the audio sensor indicating an utterance from the user. The stream of digital data may include one or more video streams. In some examples, the intelligent medical device further includes a display screen and the programming instructions are configured to cause the processor to prompt, via the display screen, the user to input an annotation. In some examples, the programming instructions are configured to flag the stream of digital data with metadata including the annotation. The one or more sensors may include multiple sensors, each sensor configured to generate a time-stamped stream of digital data.

In another aspect, the disclosure provides a method of presenting stored data for review. The method includes receiving a stream of digital data from an intelligent medical device, the stream of digital data including metadata indicating a moment in time during a medical procedure. The method further includes storing the received data and the metadata for subsequent review. The method further includes determining, based on the metadata, a start time in the received data for replaying the stream of digital data and presenting the stored digital data for review starting at a point in the stored digital data indicated by the start time.

Implementations of the disclosure may include one or more of the following optional features. In some examples, the method further includes, in response to receiving the stream of data including the metadata, transmitting a notification. In some examples, the method further includes receiving streams of digital data from a plurality of intelligent medical devices, storing the received streams of digital data, and presenting the stored streams for review starting at a point in the stored digital data indicated by the start time. The metadata may further include an annotation and the method may further include presenting the annotation. Presenting the annotation may include presenting the annotation in response to user input. In some examples, the method further includes determining, based on the metadata, a stop time in the received data for replaying the stream of digital data and stopping the presentation of the stored digital data at a point in the stored digital data indicated by the stop time. The method may further include displaying a timestamp corresponding to the point in the data stream being presented.

### BRIEF DESCRIPTION OF THE DRAWINGS

The accompanying drawings are incorporated into this document and form a part of the specification.

FIG. 1 illustrates an example environment for performing medical procedures on a subject.

FIG. 2 is a flowchart 200 of a method for flagging surgical information with metadata.

FIG. 3 illustrates an example environment for replaying stored information.

FIG. 4 is a flowchart of a method for replaying stored information.

FIG. 5 shows a block diagram of an example of internal hardware that may be used to contain or implement program instructions according to an embodiment.

In the drawings, like reference numbers generally indicate identical or similar elements. Additionally, generally, the left-most digit(s) of a reference number identifies the drawing in which the reference number first appears.

### DETAILED DESCRIPTION

This document describes system, apparatus, device, method and/or computer program product embodiments, and/or combinations and sub-combinations of any of the above, for flagging surgical information with metadata.

In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other and are not necessarily “superior” and “inferior”. Generally, similar spatial references of different aspects or components indicate similar spatial orientation and/or positioning, i.e., that each “first end” is situated on or directed towards the same end of the device. Further, the use of various spatial terminology herein should not be interpreted to limit the various insertion techniques or orientations of the implant relative to the positions in the spine.

The following terms shall have, for purposes of this application, the respective meanings set forth below:

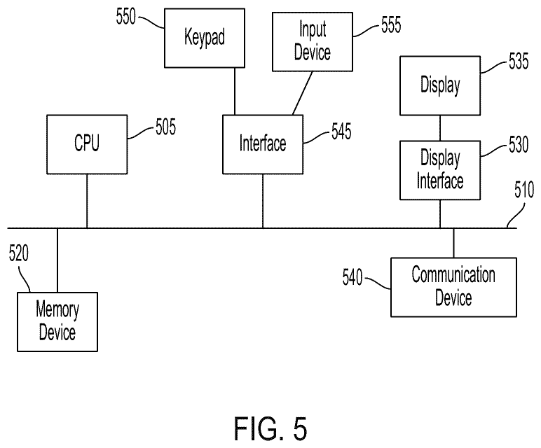

“computing device,” “electronic device,” or “computer” refers to a device or system that includes a processor and memory. Each device may have its own processor and/or memory, or the processor and/or memory may be shared with other devices as in a virtual machine or container arrangement. The memory will contain or receive programming instructions that, when executed by the processor, cause the electronic device to perform one or more operations according to the programming instructions. Examples of electronic devices include personal computers, servers, mainframes, virtual machines, containers, mobile electronic devices such as smartphones, Internet-connected wearables, tablet computers, laptop computers, and appliances and other devices that can communicate in an Internet-of-things arrangement. In a client-server arrangement, the client device and the server are electronic devices, in which the server contains instructions and/or data that the client device accesses via one or more communications links in one or more communications networks. In a virtual machine arrangement, a server may be an electronic device, and each virtual machine or container also may be considered an electronic device. In the discussion below, a client device, server device, virtual machine or container may be referred to simply as a “device” for brevity. Additional elements that may be included in electronic devices will be discussed below in the context of FIG. 5.

The terms “memory,” “computer-readable medium” and “data store” each refer to a non-transitory device on which computer-readable data, programming instructions or both are stored. Unless the context specifically states that a single device is required or that multiple devices are required, the terms “memory,” “computer-readable medium” and “data store” include both the singular and plural embodiments, as well as portions of such devices such as memory sectors.

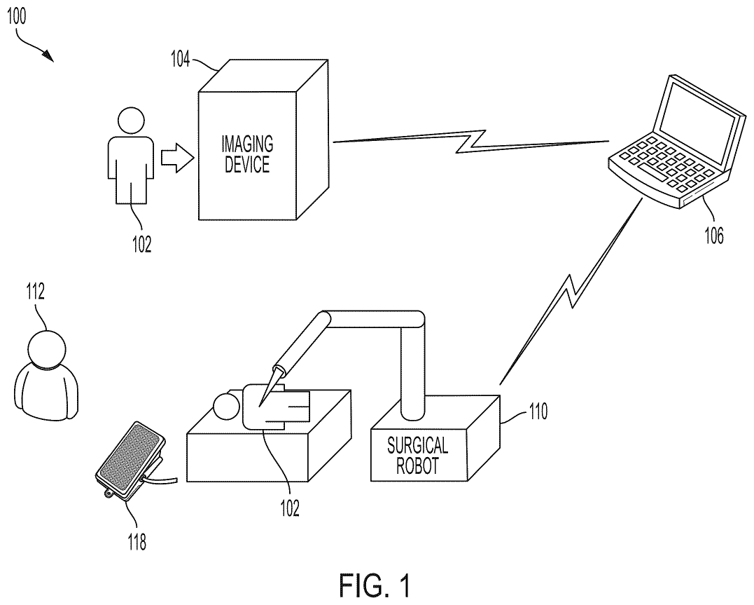

The system(s) and method(s) described in this disclosure are generally directed to flagging surgical information with metadata. FIG. 1 illustrates an example environment 100 for performing medical procedures on a subject 102, e.g., by clinician 112. The medical procedure may include an examination, treatment, surgery, etc. In some examples, the medical procedure includes imaging systems 104, such as cameras, x-ray imagers, Computed Tomography (CT) scanners, Magnetic Resonance Imaging (MRI) systems, ultrasound imagers, endoscopes, fluoroscopes, etc. The imaging devices may be independent or standalone devices, and/or the imaging systems may be a component of (e.g., incorporated into) other systems, such as image-guided surgical instruments or tools. In some examples, the medical procedure may include or involve a robotic system 110 (or other computer-enhanced system) configured to assist in various aspects of the procedure, such as positioning surgical tools or instruments, positioning the subject of the procedure, positioning medical implants prior to implantation, etc. The robotic system 110 may include position sensors for monitoring components of the robotic system, including monitoring the component's position, orientation, velocity, acceleration, and so forth, e.g., to help navigate the use of surgical tools during the procedure. The robotic system 110 may also include additional sensors configured to measure strain, stress, tissue density, temperature, or other quantity relevant for the medical procedure. As described above, the robotic system 110 may also include one or more imaging system, e.g., to capture images of the subject 102 or the subject's anatomy.

In some examples, the robotic system 110 and/or the imaging systems 104 generate digital data during operation. The digital data may include image data, sensor data, and/or other data associated with the operation of these devices. The digital data may further include timestamps or other metadata that provides information about other data (e.g., sensor/image data). The robotic system 110 and/or the imaging systems 104 may transmit (or otherwise provide) the digital data to a server 106 for preservation (e.g., in a continuous or episodic data stream). The server 106 receives and stores the generated data, and may make the stored data available for subsequent review or “playback” of the medical procedure. In some examples, the server 106 may be integrated within the robotic system 110 and/or the imaging systems 104, or may otherwise be closely associated with those systems. In other cases, the server 106 may be a remote and/or cloud-based system.

After the medical procedure is complete, the clinician 112 may access the stored data, e.g., to review a particular moment (or period of time) of interest during the procedure. In some examples, the clinician 112 may only become aware of a moment (or period of time) that should be reviewed after the procedure is complete. In other examples, the clinician 112 becomes aware while the procedure is being performed, e.g., that an aspect of the procedure has deviated from expectations in a significant way. For example, the clinician 112 may realize that the procedure cannot be performed as expected, or the clinician 112 may detect additional conditions and/or pathologies that require reevaluating and/or replanning the procedure. In some examples, the clinician 112 may become aware of an error or other off-normal incident or occurrence that will require review and analysis. The clinician 112 may want to review the incident after the procedure is complete, or even before the procedure is complete (e.g., to determine how best to complete the procedure). In some examples, the clinician 112 may want to highlight an interesting and/or challenging portion of the procedure for educational purposes, e.g., allowing others to review the portion of the procedure, e.g., to learn from the experience of the clinician 112.

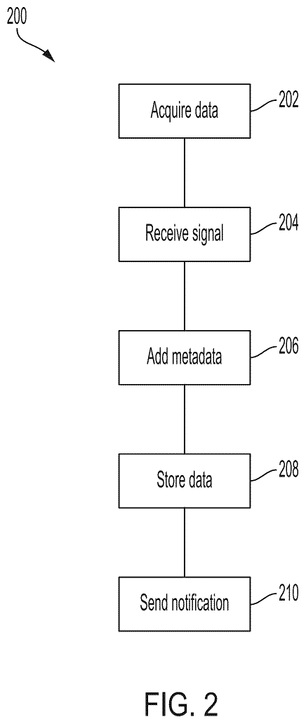

FIG. 2 is a flowchart 200 of a method for flagging surgical information with metadata. As described above, a clinician 112 may be performing a procedure (e.g., on a subject) in an environment, such as the environment of FIG. 1. For example, the clinician 112 may be performing an endoscopy or other procedure that requires careful manipulation of surgical tools, instruments, probes, sensors, or the like. In some examples, the procedure also requires the clinician 112 to carefully observe a visual display indicating the progress of the procedure. The visual display may show images, e.g., from the imaging devices 104. Alternatively (or in addition), the visual display may include raw or processed data from sensors of the surgical tools/instrument/probe. During the procedure, the clinician 112 may become aware of a significant moment (or period) that should be reviewed later (e.g., after the procedure is complete), such as an error or other off-normal incident. To facilitate identifying the significant moment in the stored data, the data source (e.g., imaging device 104, robotic system 110, or other intelligent surgical device) may insert metadata in the data stream indicating the significant moment (or period).

At step 202, the method includes acquiring data related to a medical procedure. Medical procedures may include diagnostic and/or corrective procedures, such as robotically assisted surgeries, endoscopies, ultrasound scans, x-ray (or other imaging) scans, computed tomography (CT) scans, or other procedures related to assessing or addressing a medical issue of a subject. Acquiring data may include obtaining data from an imaging or other diagnostic system, or data from a sensor associated with a surgical instrument, such as a camera, pressure sensor, position sensor, temperature sensor, tissue density sensor, or other intelligent medical device. Intelligent medical devices include imaging devices 104, robotic systems 110, and/or other computer-based systems used to assist performing the procedure, to record aspects of the procedure, or otherwise assist and/or monitor performing the procedure. As described above, intelligent medical devices may generate digital information that may be stored for later playback and/or review. At step 204, the method includes receiving, by an intelligent medical device, a signal indicating a moment for later review. Receiving the signal may include detecting a button press, knob turn, or other interaction with a control panel (physical or virtual) of the intelligent medical device, if so equipped. As described above, the procedure may require careful manipulation of surgical tools and careful observation of visual display screens. In such circumstances, it may be difficult for the clinician 112 to interact with a control panel while performing the procedure. That is, the clinician's hand may be fully occupied and/or the procedure may require the clinician 112 to continually monitor one or more visual display screens. In this case, it may be difficult or impossible for the clinician 112 to access a control panel. To address this situation, the intelligent medical device may include a dedicated input device, such as a floor pedal 118, configured to receive the signal without significantly interfering with the procedure. In some examples, the floor pedal 118 may physically move in response to input (e.g., pressure) from the clinician's foot. In other examples, the foot pedal 118 may sense increased pressure, e.g., that exceeds a pressure threshold, even without physically moving. The intelligent medical device may include a virtual foot pedal, e.g., sensing motion of the clinician's foot. For example, the intelligent medical device may include one or more cameras configured to detect foot motion, e.g., from side to side, or up and down. Alternatively, or in addition, the intelligent medical device may include a button on a portion of the device that is manipulated by the clinician 112. For example, vessel-sealing devices, staplers, and other medical devices may include a pistol-like grip. The pistol grip may provide a convenient location for a finger-operated switch (e.g. thumb switch). The clinician 112 may readily access the grip-mounted switch without having to change hand positions or otherwise interrupt or interfere with performing the procedure. In some examples, the switch is a physical switch, such as a push button or rocker switch. In other cases, the switch may be touch-sensitive, e.g., sensing a change in capacitance due to touch.

In some examples, receiving the signal includes recognizing a signal in an utterance of the clinician 112. For example, the intelligent medical device may be configured to recognize one or more keywords, phrases, or other speech uttered by the clinician 112. The speech may include a command, such as “Record,” potentially preceded by a word or phrase indicated that a command will follow, such as a “wake word.” In this case, the intelligent medical device may ignore commands unless they are preceded by the wake word/phrase. In some examples, the intelligent medical device may apply natural language processing to speech uttered by the clinician 112 to recognize the signal to flag a moment as a moment of interest. In this example, the intelligent medical device may recognize a wide variety of utterances as indicating a moment of interest, including, e.g., exclamations and/or outbursts.

In some examples, receiving the signal includes recognizing a signal in combinations of actions and/or utterances. For example, the intelligent medical device may ignore button presses and/or pedal presses unless they are preceded by the wake word/phrase. Conversely, the intelligent medical device may ignore utterances unless they are preceded by (or contemporaneous with) a button and/or pedal press. The intelligent medical device may be configured to recognize other combinations actions and/or utterances as a signal received from the user as well.

In some examples, the intelligent medical device may learn over time to recognize signals in particular combinations of actions and/or utterances of a clinician 112, e.g., using machine learning techniques. For example, based on previous interactions with a clinician 112, the intelligent medical device may identify patterns of actions and/or utterances that often precede or accompany an explicit command, from the clinician 112, to flag a moment of interest. Subsequently, the intelligent medical device may recognize the pattern of actions and/or utterances as a signal and may, in response, automatically flag the moment as a moment of interest. In some examples, the intelligent medical device may ask the clinician 112 for confirmation that the moment should be flagged. If the clinician 112 indicates that the intelligent medical device flagged the moment in error, the intelligent medical device may “unflag” the moment and update its training.

At step 206, the method includes adding metadata to the digital information generated by the intelligent medical device in response to receiving the signal. In some examples, the metadata includes a flag or marker indicating a moment of interest (e.g., as indicated by the clinician 112). The flag or marker may also include a timestamp indicating when the intelligent medical device received the signal. In some examples, the metadata indicates a window of time, e.g., a starting timestamp (occurring shortly before the intelligent medical device receives the signal) and an ending timestamp (occurring at a period of time afterwards). In an example, the window of time may start five seconds prior to receiving the signal and continue for thirty seconds after receiving the signal. Other time windows are also within the scope of this disclosure, including time windows that occur completely before or completely after the moment of interest. In some examples, the metadata also includes a separate, parallel data stream, e.g., including data acquired during the time window. That is, using the example time window described above, when the intelligent medical device receives the signal, it may duplicate the previous five seconds of information, acquire an additional thirty seconds of information, and add the separate thirty-five seconds of information (along with its associated time information) as metadata to the generated digital information. In some examples, the intelligent medical device generates separate information streams, e.g., from multiple imaging devices and/or multiple sensor devices. In these examples, the intelligent medical device may add metadata to each of these streams using the same manner, or the intelligent medical device may add metadata to one or more information streams in different manners. For example, the metadata added to some data streams may include only a timestamp, and the metadata added to other data streams may also include a separate, parallel data stream for the duration of a time window.

In some examples, the intelligent medical device may provide a user interface, e.g., via a computer display screen. In response to receiving the signal, the intelligent medical device may provide the opportunity to input additional information, such as notes describing the reason for flagging the surgical information for later review. In some examples, the intelligent medical device may present an input window capable of receiving text input. The input text may be added to the metadata and stored with the digital information for later review. In some examples, the intelligent medical device may automatically begin recording audio input in response to receiving the signal, so that the clinician 112 may input additional information orally (e.g., if the intelligent medical device lacks a computer screen). To receive audio input, the intelligent medical device may include a wired or wireless microphone, such as wireless earphones having an integrated microphone. The intelligent medical device may indicate, using an appropriate manner, the opportunity to input additional information. For example, the intelligent medical device may provide an audio announcement (e.g., via the earphones) indicating the imminent beginning of recording of audio input, or the intelligent medical device may make a beeping sound or flash a light, or illuminate text, or otherwise indicate the imminent beginning and/or ending of audio recording. In some examples, the intelligent medical device includes speech-to-text conversion, so that audio input is converted to text. The intelligent medical device may add the text to the metadata instead of or in addition to the audio input from which the text was converted.

At step 208, the method includes storing the information and the metadata (e.g., on server 106) for subsequent review. In some examples, the intelligent medical device is configured to display or replay the stored information, allowing the clinician 112 to review the procedure. In other examples, the clinician 112 may review the stored information on a separate system, e.g., a device configured to replay information stored (e.g., by intelligent medical devices) during a procedure. In some examples, the method also includes, at step 210, transmitting a notification to the user indicating that flagged data has been stored on the server. For example, the method may include sending an e-mail, text message, mobile device alert, or other form of notification. The notification may include a instructions for accessing the stored information and/or a reference to the information, such as a hyperlink, that the user may access to view or replay the information.

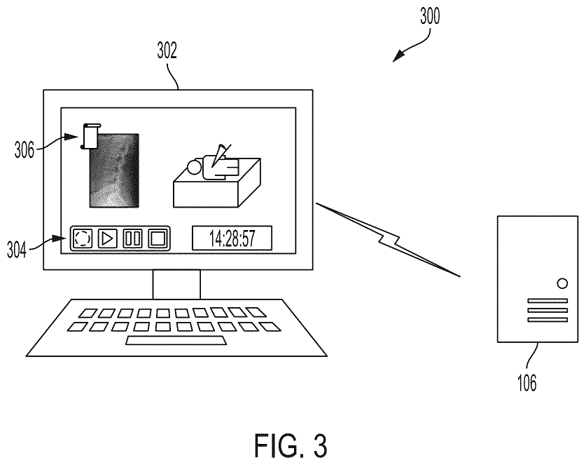

Referring to FIG. 3, an environment 300 for replaying stored information is shown. The environment 300 may include a replay system 302 configured to display stored information from one or more intelligent medical devices, imaging devices 104, and/or other sources of digital data. In some examples, the replay system 302 is configured to display information from one source of digital data, or to display information from one source of digital data at a time. For example, the replay system 302 may be the source of digital data. That is, an intelligent medical device may be capable of replaying data generated by the medical device (and stored on the medical device or a remote server 106). In other examples, the replay system 302 may display information from a variety of sources and may display the information from different sources at the same time, e.g., on different portions of a display screen. For example, the display screen may be subdivided into separate portions, each portion displaying information from a different source. In the case of imaging systems, the display screen may be divided into a grid of tiles, each tile displaying a separate video stream. The replay/display system 302 may be configured to synchronize the information from each source, e.g., by aligning data and/or video according to associated time stamps or other time information associated with the data or video. In this way, external data (e.g., video streams from devices that are not integrated with the intelligent medical devices) may be displayed along with simultaneously acquired data from intelligent medical devices.

In some examples, the display system 302 includes an interface 304 which displays the timestamp(s) associates with the data/video and which may allow users to advance or reverse time-sequenced data/video. In some examples, the display system 302 may be configured to recognize metadata included in the recorded information (e.g., recorded data/video) and to display or replay the portion of the recorded information indicated by the metadata. In some examples, the interface 304 is further configured to display the additional information, e.g., notes describing the reason for flagging the surgical information, or to replay additional information recorded (e.g., in audio form) in response to flagging the surgical information. For example, the display system 302 may display an indicator 306, such as an icon, when additional information is available. In some examples, the icon may indicate the nature of the additional information (e.g., text, audio, etc.). By selecting the icon, a user may cause the display system 302 to display, replay, print, or otherwise make the additional information available to the user.

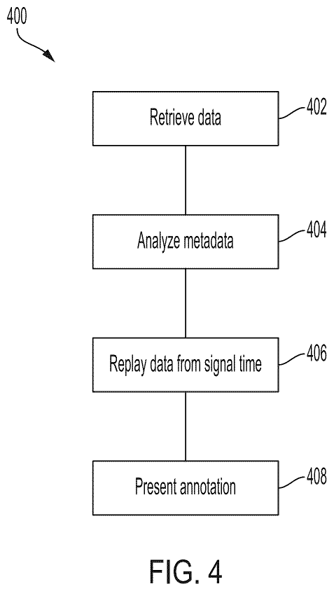

FIG. 4 is a flowchart 400 of a method for replaying surgical information flagged with metadata. At step 402, the method includes retrieving the stored information, e.g., the data stream(s) from one or more intelligent medical devices. As described above, the stored information may include one or more time-stamped data from sensors, cameras, and/or other sources of digital data acquired during a medical procedure and flagged by a user for review and/or replay. The stored information may include metadata based on a moment in time (e.g., during the medical procedure). For example, the metadata may include a timestamp indicating when a signal was received by the intelligent medical device, or a window of time, such as from five seconds before the moment until 30 seconds after the moment. At step 404, the method includes analyzing the metadata to determine a starting time for reviewing or replaying the stored information. In some examples, the starting time is the time indicated (by the metadata) as the start of a window of time. At step 406, the method includes presenting the stored information for review starting at the point of time indicated by the metadata (e.g., the beginning of the time window). In some examples, the stored information includes one or more time-stamped video streams. The method may include displaying the time-stamped video streams (e.g., replayed at a conventional playback speed) and the associated time stamp. At step 408, the method may optionally include presenting annotations associated with the stored information, e.g., annotations provided by the user in response to signaling the intelligent medical device(s) to flag a moment in time. The annotation may have been entered by the user in response to a prompt displayed to the user, or in recorded by the user, e.g., after signaling the intelligent medical device or in response to an audio prompt for annotation, e.g., from the intelligent medical device. The method may include displaying text-based annotation in text form, or as audio (e.g., after conversion from text). Similarly, the method may include presenting audio-based annotation in audio form, or in text form (e.g., after conversion from audio). The method may include presenting the annotation automatically and in parallel with replaying or displaying the stored information, or the method may include presenting the annotation in response to a user action, such as selecting indicator 306.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable gage/logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

FIG. 5 illustrates example hardware that may be used to contain or implement program instructions. A bus 510 serves as the main information highway interconnecting the other illustrated components of the hardware. Central Processing Unit (CPU) 505 is the central processing unit of the system, performing calculations and logic operations required to execute a program. CPU 505, alone or in conjunction with one or more of the other elements disclosed in FIG. 5, is an example of a processor as such term is used within this disclosure. Read only memory (ROM) and random-access memory (RAM) constitute examples of non-transitory computer-readable storage media 520, memory devices or data stores as such terms are used within this disclosure.

Program instructions, software or interactive modules for providing the interface and performing any querying or analysis associated with one or more data sets may be stored in the memory device 520. Optionally, the program instructions may be stored on a tangible, non-transitory computer-readable medium such as a compact disk, a digital disk, flash memory, a memory card, a universal serial bus (USB) drive, an optical disc storage medium and/or other recording medium.

An optional display interface 530 may permit information from the bus 510 to be displayed on the display 535 in audio, visual, graphic or alphanumeric format. Communication with external devices may occur using various communication ports 540. A communication port 540 may be attached to a communications network, such as the Internet or an intranet.

The hardware may also include an interface 545 which allows for receipt of data from input devices such as a keypad 550 or other input device 555 such as a touch screen, a remote control, a pointing device, a video input device and/or an audio input device.

Based on the teachings contained in this disclosure, it will be apparent to persons skilled in the relevant art(s) how to make and use embodiments of this disclosure using data processing devices, computer systems and/or computer architectures other than that shown in FIG. 5. In particular, embodiments can operate with software, hardware, and/or operating system implementations other than those described in this document.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes example embodiments for example fields and applications, it should be understood that the disclosure is not limited to the disclosed examples. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described in this document. Further, embodiments (whether or not explicitly described) have significant utility to fields and applications beyond the examples described in this document.

Embodiments have been described in this document with the aid of functional building blocks illustrating the implementation of specified functions and relationships. The boundaries of these functional building blocks have been arbitrarily defined in this document for the convenience of the description. Alternate boundaries can be defined as long as the specified functions and relationships (or their equivalents) are appropriately performed. Also, alternative embodiments can perform functional blocks, steps, operations, methods, etc. using orderings different than those described in in this document.

The features from different embodiments disclosed herein may be freely combined. For example, one or more features from a method embodiment may be combined with any of the system or product embodiments. Similarly, features from a system or product embodiment may be combined with any of the method embodiments herein disclosed.

References in this document to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described in this document. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described example embodiments, but should be defined only in accordance with the following claims and their equivalents.

1. A method of flagging surgical information, comprising, by a processor of an intelligent medical device:
acquiring data from one or more sensors of the intelligent medical device;
receiving a signal from a user, the signal indicating a moment in time during a medical procedure;
flagging the acquired data with metadata based on the time of the received signal; and
storing the acquired data and the metadata on a server.

2. The method of claim 1, wherein acquiring the data from the one or more sensors comprises acquiring a stream of data from the one or more sensors of the intelligent medical device.

3. The method of claim 1, wherein acquiring the data from the one or more sensors comprises acquiring the data from a plurality of sensors.

4. The method of claim 1, wherein receiving the signal from the user comprises receiving input from a floor pedal.

5. The method of claim 1, wherein flagging the acquired data with metadata comprises flagging the acquired data with metadata indicating a start time and a stop time based on the time of the received signal.

6. The method of claim 1, further comprising:
retrieving the stored data; and
presenting the stored data for review based on the metadata.

7. An intelligent medical device comprising:
one or more sensors configured to generate a stream of digital data during a medical procedure;
an input device; and
a processor and a computer-readable memory containing programming instructions that are configured to, when executed by the processor, cause the processor to:
receive a signal from the input device;
flag the stream of digital data with metadata indicating a time of the received signal; and
store the acquired data and the metadata on a server for subsequent review.

8. The intelligent medical device of claim 7, wherein the one or more sensors comprise a camera.

9. The intelligent medical device of claim 7, further comprising a surgical robot.

10. The intelligent medical device of claim 7, wherein the input device comprises an audio sensor, wherein the programming instructions that are configured to cause the processor to receive the signal comprise instructions that are configured to cause the processor to receive input from the audio sensor indicating an utterance from a user.

11. The intelligent medical device of claim 7, wherein the stream of digital data comprises one or more video streams.

12. The intelligent medical device of claim 7, further comprising a display screen, wherein the programming instructions that are configured to cause the processor to flag the stream of digital data with metadata comprise programming instructions that are configured to cause the processor to:
prompt, via the display screen, a user to input an annotation; and
flag the stream of digital data with metadata including the annotation.

13. The intelligent medical device of claim 7, wherein the one or more sensors comprise a plurality of sensors, each sensor of the plurality of sensors configured to generate a time-stamped stream of digital data.

14. A method of presenting stored data for review, the method comprising:
receiving a stream of digital data from an intelligent medical device, the stream of digital data comprising metadata indicating a moment in time during a medical procedure;
storing the received data and the metadata for subsequent review;
determining, based on the metadata, a start time in the received data for replaying the stream of digital data; and
presenting the stored digital data for review starting at a point in the stored digital data indicated by the start time.

15. The method of claim 14, further comprising, in response to receiving the stream of digital data comprising the metadata, transmitting a notification.

16. The method of claim 14, further comprising:
receiving streams of digital data from a plurality of intelligent medical devices;
storing the received streams of digital data; and
presenting the stored streams for review starting at the point in the stored digital data indicated by the start time.

17. The method of claim 14, wherein the metadata further comprises an annotation and the method further comprises presenting the annotation.

18. The method of claim 17, wherein presenting the annotation comprises presenting the annotation in response to user input.

19. The method of claim 14, further comprising:
determining, based on the metadata, a stop time in the received data for replaying the stream of digital data; and
stopping the presentation of the stored digital data at a point in the stored digital data indicated by the stop time.

20. The method of claim 14, further comprising displaying a timestamp corresponding to the point in the data stream being presented.
